(19) United States
(12) Patent Application Publication
(10) Pub. No.: US 2018/0072974 A1
(43) Pub. Date: Mar. 15, 2018

(54) STAND-UP GUSSETED FILTER BAG

(71) Applicant: 3M INNOVATIVE PROPERTIES COMPANY, ST. PAUL, MN (US)

(72) Inventor: PATRICK R. McKELLIPS, LAKELAND, MN (US)

(21) Appl. No.: 15/563,695

(22) PCT Filed: Apr. 6, 2016
(86) PCT No.: PCT/US2016/026104

§ 371 Date: Oct. 2, 2017

Related U.S. Application Data
(60) Provisional application No. 62/144,761, filed on Apr. 8, 2015.

(51) Int. Cl. C12M 1/00; B65D 33/00; B65D 33/30; C12N 1/20

(52) U.S. Cl. CPC C12M 23/14; B65D 33/007; B31B 70/812; C12N 1/20; C12M 29/04; B65D 33/30

(57) ABSTRACT

Gusseted filter bag capable of flexing between a flat configuration and an open configuration.

### TECHNICAL FIELD

The present disclosure relates to a bag containing a built-in filter.

### BACKGROUND

Liquid growth media have been used to culture test samples. The containers used for such culturing may not be convenient. A more convenient and easier to use container is needed.

### SUMMARY

A bag can comprise a first side made of a flexible, water impermeable polymeric material; a second side made of a flexible, water impermeable material, the first side and second side being attached at the edges and at an openable top; a flexible gusseted bottom having a concave upper portion, wherein the bottom is capable of flexing between a flat configuration and an open configuration; a filter positioned inside the bag, the filter having a convex lower portion, wherein the filter is affixed to the first side and the second side, and wherein the convex lower portion of the filter is affixed to the concave upper portion of the bottom; and a closing mechanism for closing the openable top.

A method of preparing a culture sample can comprise converting a bag as described herein to the open configuration, thereby forming two compartments, the first compartment defined by the first side and the filter and the second compartment defined by the second side and the filter; opening the sealed top; and placing a test sample in one of the first compartment or the second compartment such that the test sample contacts the liquid growth media.

Related products and methods are also disclosed.

### BRIEF DESCRIPTION OF THE DRAWINGS

FIG. 1 is a front view of a filter bag in the flat configuration.

FIG. 2 is a profile view of a filter bag in the open configuration.

FIG. 2a is a cut-away side view of the filter bag of FIG. 2.

FIG. 3 is a profile view of a filter bag in the open configuration.

FIG. 4 is a front view of a filter bag in the flat configuration.

FIG. 5 is a profile view of a filter bag in the open configuration.

FIG. 6 is a profile view of a filter bag in the open configuration.

FIG. 6a is a cut-away side view of the filter bag of FIG. 6.

FIG. 7 is a profile view of a filter bag.

### DETAILED DESCRIPTION

Throughout this disclosure, singular forms such as “a,” “an,” and “the” are often used for convenience; however, it should be understood that the singular forms are meant to include the plural unless the singular alone is explicitly specified or is clearly indicated by the context.

A bag can contain a first side and a second side, each of which can be made of a flexible, water impermeable material. The first side and the second side can be attached at the side edges and at a sealed top. The bottom of the bag can be gusseted, for example, with two gussets, such that the bottom is capable of flexing between a flat configuration and an open configuration. The bottom can be made from the flexible, water impermeable material. The bottom can have a concave portion. This shape can allow liquid to pool in the concave portion, which can be advantageous for facilitating one or more of collection and removal of the liquid; it can also be advantageous to facilitate contact of a test sample with the liquid.

A filter can be positioned in the bag between the first side and the second side. The filter can be attached to the first and second sides at the edges of the bag. This attachment is typically by way of, for example, welding, although lamination using adhesives is also possible.

The top edges of the two flexible sides can meet at the top of the bag to form an openable top. The filter typically does not extend all the way to the openable top, and instead is shorter than both of the two flexible sides to leave a vacant space between the top of the filter and the openable top. A closing mechanism can be present to allow a user to close the openable top after it is opened.

The first side and the second side can be made out of any suitable material that is flexible and impermeable to water. Although some flexibility is needed, suitable materials will typically be sufficiently stiff so as to allow the bag to stand up on a horizontal surface when the gusseted bottom is in an open configuration. In most cases, a polymeric material is used. Suitable polymeric materials include one or more of nylon, polyethylene, and polypropylene. Low density polyethylene is common, particularly linear low density polyethylene. Laminates of one or more layers of the above mentioned materials can also be used. An exemplary laminate is a nylon-linear low density polyethylene laminate. In such laminates, the nylon is often on the exterior of the bag and the linear low density polyethylene is often on the interior of the bag.

The first side and the second side can be any suitable thickness. The thickness is often from about 30 microns to about 150 microns. When a laminate is used, the inner layer is typically thicker than the outer layer. In many cases, the inner layer is from about 2 to about 4 times the thickness of the outer layer. One exemplary laminate has an approximately 15 micron thick outer layer and an approximately 50 micron thick inner layer. In that case, the outer layer can be made of nylon and the inner layer can be made of low density polyethylene, such as linear low density polyethylene.

The first side and the second side can be joined together at the edges; the filter is commonly joined to the two sides at the edges as well. The first side and second side can also be joined together at the top of the bag to form an openable top. The openable top can be perforated so that it can be readily opened by hand tearing. Another way to make the openable top openable by hand is to use a two layer laminate material, typically a linear low density polyethylene/nylon laminate, that is partially scored. Partially scored laminates typically have a score line that penetrates through the outer layer of the laminate but does not break, penetrate, or perforate the inner layer of the laminate. This allows for easy opening of the bag while still maintaining a sterile bag interior, because the interior layer of the laminate does remains fully intact without any scores, perforations, or breaks. One or more notches aligned with the scoring can be present on the edges of the bag. Such notches can further facilitate easy opening of the bag.

It is also possible that the openable top can be configured without perforations or scoring, for example partial scoring as discussed herein, such that a cutting element, for example scissors or a knife, is needed to open it. Indicia such as indentations, a dotted line, one or more pictures of scissors, the words “open,” “open here,” or the like, can be present on or near the openable top for the convenience of the user.

The gusseted bottom typically has two triangular or semi-circular portions that can fold into a flat configuration or an open configuration. The gusseted bottom can be attached to the two sides by any suitable method, with welding being the most common. The gusseted bottom can be made of any suitable material, typically a flexible and water impermeable material. In most cases, a polymeric material is used. Suitable polymeric materials include one or more of nylon, polyethylene, and polypropylene. Low density polyethylene is common, particularly linear low density polyethylene. Laminates of one or more layers of the above mentioned materials can also be used. An exemplary laminate is a nylon-linear low density polyethylene laminate. In such laminates, the nylon is often on the exterior of the bag and the linear low density polyethylene is often on the interior of the bag.

The interior of the gusseted bottom has a concave upper portion, such that the center portion of the bag is deeper than the edges. This shape allows a liquid to pool in the center of the bag, which in turn can facilitate contact of a liquid, such as growth medium, with a test sample. The shape can also facilitate removal of liquid, for example, with a pipette.

The filter is typically affixed to each of the two sides by welding. In most cases, the filter is shorter than the two sides such that the top of the filter does not extend all the way to the openable top. In such cases, there is a vacant space between the top of the filter and the openable top when the bag is standing upright.

The filter can made of any suitable material. Nylon and polyethylene are often used, with polyethylene being most common. A combination of more than one polymer can also be employed. Such combinations can be blends. Suitable blends include a blend of polyethylene and polypropylene, a blend of polyethylene and polyethylene terephthalate, and a blend of polypropylene and polyethylene terephthalate. Blends of polyethylene and polypropylene are common. In such blends, the ratio of polyethylene to polypropylene can be any suitable ratio, but is most commonly 1:1.

In some cases, the filter is prepared from biocomponent fibers, such as sheath/core biocomponent fibers. Such fibers often include a polyethylene sheath and polypropylene core, a polyethylene sheath and polyethylene terephthalate core, or a polypropylene sheath and polyethylene terephthalate core. Such fibers can be a woven or non-woven.

Appertured films, such as those made from non-woven materials, are commonly used as filters. Any type of apertured film can be used for the filter. Some known apertured films are described in WO 93/15701 and U.S. Pat. No. 6,022,607. Typically, an aperture film has a plurality of protrusions that protrude from the same side of the film. The protrusions can be cup shaped, v-shaped, or the like, and are in many cases distributed in a repeating pattern on the film. The protrusions typically have holes, known as apertures, which can be in any portion of the protrusion but are typically in the portion that is farthest from the film. One suitable aperture film is available under the trade designation 40 HEX LDPE (X-6582), which is commercially available from TREDEGAR FILM PRODUCTS.

The filter pores, or apertures in the case of an aperture film, are sufficiently small such that particles of a test sample, such as a piece of food, for example meat, cannot pass through. Often, the filter pores or apertures are from about 0.1 microns to about 100 microns in size, although smaller or larger pores are possible depending on the intended use.

The bottom of the filter can have a convex shape to fit in the concave upper portion of the gusseted bottom. The bottom of the filter can be attached to the gusseted bottom, for example, by welding. The filter is typically configured such that when the gusseted bottom is the open configuration the filter is closer to the first side than it is to the second side. This configuration can facilitate placing a test sample or pipette into the bag.

The bag typically comprises a closing mechanism. The closing mechanism allows the sealable top to be closed after the seal is broken to open the bag. Any closing mechanism for a bag can be used. One suitable closing mechanism described in U.S. Pat. No. 6,273,608 uses metal strips. Another closing mechanism is commercially available in bags sold under the trade designation “ZIPLOC.” Yet another the closing mechanism comprises a piece of repositionable tape attached to the bag. The repositionable tape can be removed from the bag, the top of the bag rolled up to close it, and then the tape placed over the rolled portion to keep the bag closed. The most common closing mechanism is a tie made of a flexible wire or a flexible wire covered with paper or plastic. Such ties are known in the art, and are disclosed in, for example, U.S. Pat. No. 6,585,413 and U.S. Pat. No. 6,273,608, both of which are hereby incorporated by reference for their disclosure of closure systems. When not in use, the tie typically extends beyond the sides of the bag. In use, the top of the bag is rolled up to close the bag and the tie bent around the rolled portion to keep it in place.

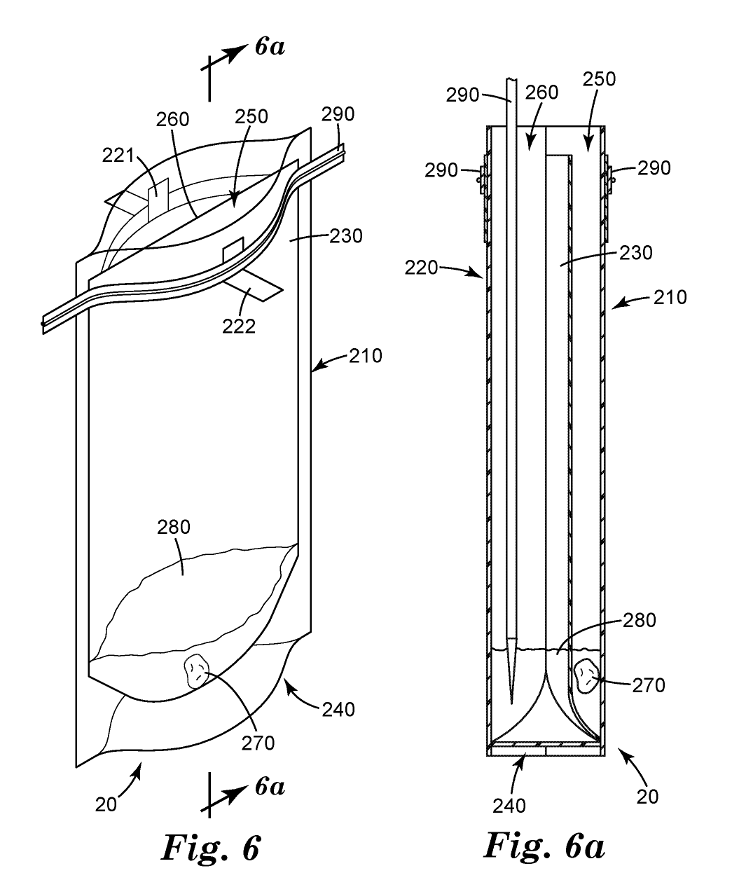

The bag can also include one or more pull tabs on the exterior of the bag. When included, two pull tabs are typically used. In the most common configuration, a first pull tab is affixed to an exterior portion of the first side of the bag, and a second pull tab is affixed to an exterior portion of the second side of the bag. The one or more tabs are typically made of a plastic material. The one or more pull tabs can be affixed to the bag by any suitable means, such as welding or lamination with adhesive. In use, the bag can be opened by pulling the pull tabs in opposite direction. The pulling force can open the openable top. Pull tabs suitable for this purpose are described in U.S. Pat. No. 658,413, for example, as item number 30 in FIG. 6 and the description thereof. U.S. Pat. No. 6,658,413 is incorporated by reference for its disclosure of pull tabs and their use for opening a bag.

The bag is often pre-filled with a liquid growth medium. Any suitable type of growth medium can be used, depending on the desired application. Suitable growth media include nutrient broths, minimal media, selective media, differential media, transport media, enriched media, and the like. Other examples of suitable liquid growth media include lactose broth, tryptic soy broth, buffered peptone water, and UVM modified lysteria enrichment broth.

The volume of liquid growth medium will depend on the size of the bag and intended use. When the bag is to be used for culturing a test sample, sufficient liquid growth medium to contact, for example, to submerge, the test sample is typically used. In a typical case, about 150 mL to about 300 mL, such as about 225 mL, of liquid growth media is used. For a typically sized bag, which is about 10 to about 25 cm wide and about 15 to about 40 cm high, such as about 15 cm wide and about 27 cm high, in the closed configuration, this amount of liquid growth media is sufficient for good contact with a typical test sample having a mass between about 5 and about 50 g, such as about 25 g.

The bag can be made by any suitable method. A variety of methods are known in the art. One suitable method involves using a machine of the type described in U.S. Pat. No. 7,238,253 and the methods described therein. After manufacture, the bag can be sterilized. Sterilizing the bag can be accomplished by any suitable method, but is most commonly performed by gamma irradiation. Thus, the materials used to make the bag are, in most cases, chemically and physically stable under sterilizing does of gamma radiation.

In use, a bag as described herein can be converted to the open configuration thereby forming two compartments. The first compartment is defined by the filter and the first side, and the second compartment is defined by the filter and the second side. The sealed top can then be opened, for example by tearing by hand or using a cutting element, and a test sample placed in one of the first compartment or the second compartment. If the bag is pre-filled with liquid growth media then the sample is placed such that it contacts the liquid growth medium; otherwise, liquid growth medium can be added either before or after adding the sample.

The test sample can be any sample for culturing or testing for microorganism growth. Typical test samples are food items or pieces of food items, for example, pieces of fruit, vegetable, meat, bread, baked goods, and the like. Meat samples are common.

The top of the bag can then be closed using the closing mechanism. The test sample can be allowed to remain in the bag for sufficient time to culture the test sample, for example, for a sufficient time to allow microorganisms to form one or more colonies in the liquid growth medium. Culturing can take place under any suitable conditions, which can be ambient temperature, refrigerator temperature (about 40° F.), freezer temperature (about 30° F.), or elevated temperature.

After a sufficient period of time, which in some cases is as short as a few minutes and in other cases as long as one week, has elapsed, some of the liquid growth medium can be removed from the bag for further testing. The liquid growth medium is typically removed by pipette, which can be placed in whichever compartment does not contain the test sample. This facilitates removal of the liquid growth medium because particles of the test sample cannot pass through the filter and clog the pipette. The concave shape of the bag's bottom also facilitates removal by pipette because this shape allows for pooling of the liquid growth medium at the bottom of the bag; thus, the shape works to facilitate pipetting in the same way as the shape of an Eppendorf tube.

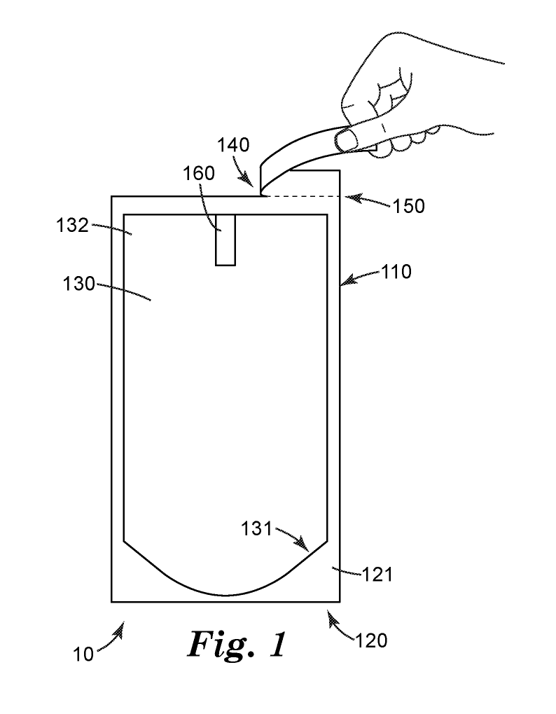

Turning to the Figures, FIG. 1 is a side view of bag 10 wherein first side 110 is visible (the second side is not visible in this view). Gusseted bottom 120 is in the flat configuration, and includes concave upper portion 121. Filter 130 is inside bag 10 and is attached to the edges of the first side (and the second side, which is not visible). Filter 130 includes convex lower portion 131 that is attached to gusseted bottom 120 at concave upper portion 121. Openable top 140 is, in this case, being opened by tearing the scored portion 150 of openable top 140. First pull tab 160 is attached to first side 110, where it could be used in conjunction with a second pull tab (not shown) to open opentable top 140. Upper portion 132 of filter 130 is below openable top 140, creating some vacant space between those two elements.

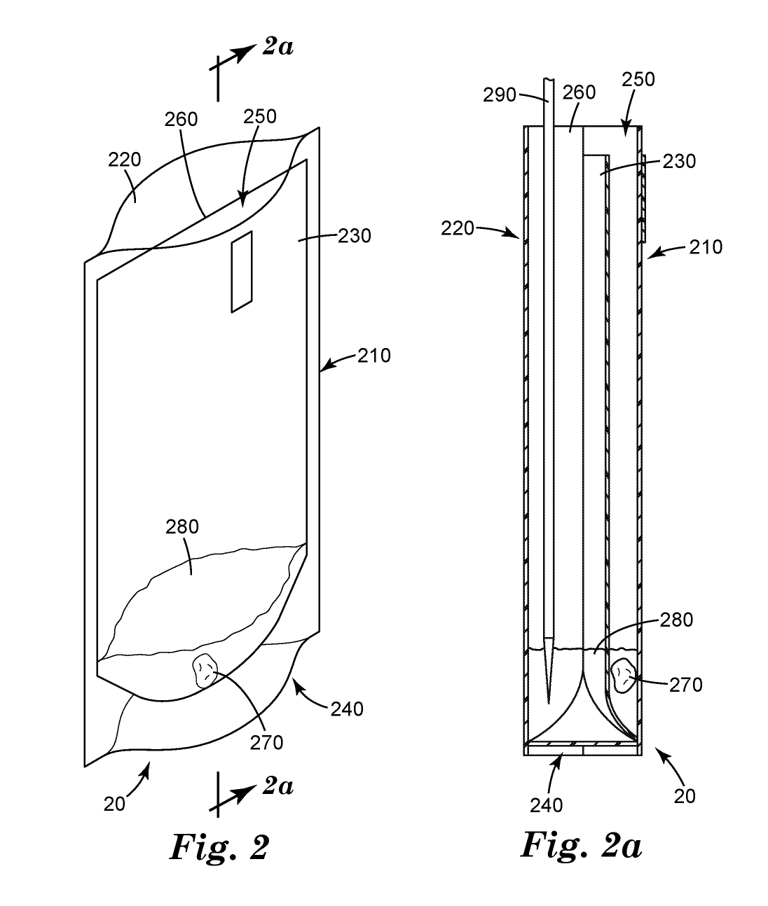

FIG. 2 is a profile view of bag 20 in an open configuration. In this Figure, first side 210 and second side 220 are visible. Filter 230 is attached to first side 210, second side 220, and gusseted bottom 240 to create a first compartment 250 defined by filter 230 and first side 210 and second compartment 260 defined by second side 220 and filter 230. Test sample 270 is in first compartment 250 and in contact with growth medium 280.

FIG. 2a is a cut-away side view of bag 20. In this view, test sample 270 resides in first compartment 250 between filter 230 and first side 210. Pipette 290 is in second compartment 260 to remove some liquid growth medium 280 for further testing.

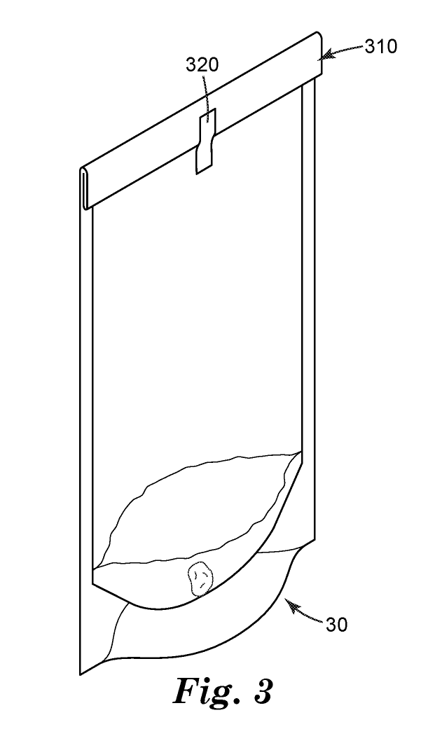

FIG. 3 shows bag 30 in an open configuration. Openable top 310 has been opened and folded over itself, and is closed with repositionable tape 320.

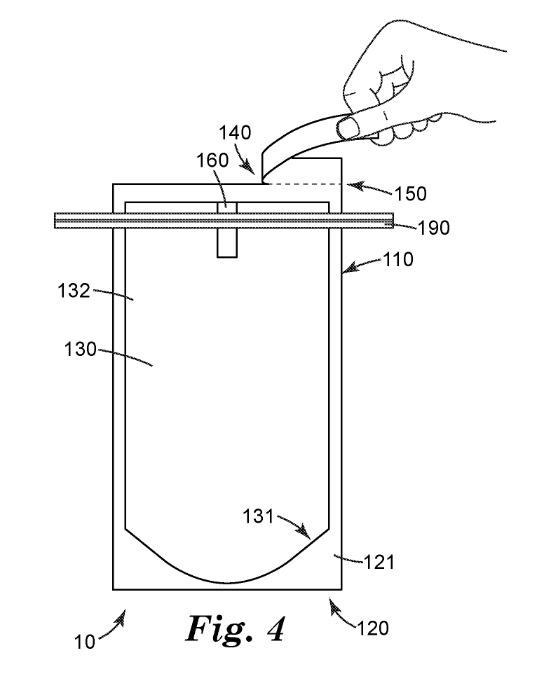

FIG. 4 shows another configuration of bag 10. In this configuration, bag 10 includes tie 190, which is affixed to bag 10 just below openable top 150.

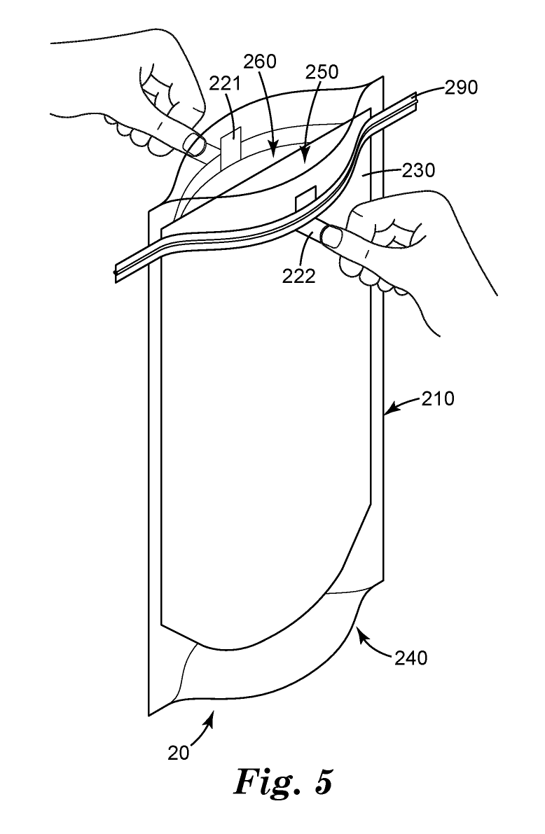

FIG. 5 shows another configuration of bag 20. In this configuration, bag 20 includes first tab 221, second tab 222, and tie 290. In operation, pulling first tab 221 and second tab 222 aseptically opens bag 20.

FIG. 6 is a profile view of bag 20 in the open configuration, wherein bag 20 opened by pulling first tab 221 and second tab 222.

FIG. 6a is a cut-away side view of bag 20.

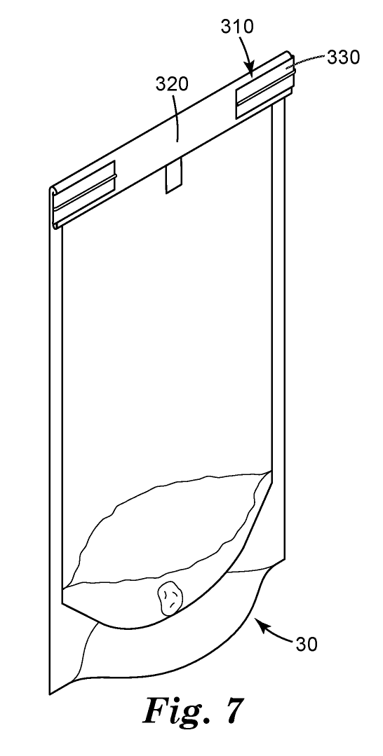

FIG. 7 is another configuration of bag 30, which has been closed by securing rolled up openable top 310 with wire 330.

1. A bag comprising
a first side made of a flexible, water impermeable polymeric material;
a second side made of a flexible, water impermeable material, the first side and second side being attached at the edges and at an openable top;
a flexible gusseted bottom having a concave upper portion and a bottom that is capable of flexing between a flat configuration and an open configuration;
a filter positioned inside the bag, the filter having a convex lower portion, wherein the filter is affixed to the first side and the second side, and wherein the convex lower portion of the filter is affixed to the concave upper portion of the bottom; and
a closing mechanism for closing the openable top.

2. The bag of claim 1, wherein the bag is capable of standing upright on a horizontal surface when the bottom is in an open configuration.

3. The bag of claim 1, further comprising scored portions on the openable top.

4. The bag of claim 1, wherein the filter is configured such that when the bag is in an open configuration the filter is closer to the first side than the second side.

5. The bag of claim 1, further comprising liquid growth medium inside the bag.

6. The bag of claim 1, wherein the closing mechanism comprises a wire.

7. The bag of claim 1, wherein the first side, the second side, and the bottom of the bag comprise at least one polymer selected from nylon, polyethylene, and polypropylene.

8. The bag of claim 7, wherein the at least one polymer comprises linear low density polyethylene.

9. The bag of claim 7, wherein the at least one polymer comprises nylon and polyethylene.

10. The bag of claim 9, wherein the nylon and polyethylene are laminated together such that a layer of nylon is on a bag exterior and a layer of polyethylene is on a bag interior.

11. The bag of claim 10, wherein the nylon on the bag exterior is scored at the openable top and the polyethylene on the bag interior is not scored.

12. The bag of claim 1, wherein the filter is an apertured filter.

13. The bag of claim 1, wherein the filter comprises nylon.

14. The bag of claim 1, wherein the bag comprises a vacant space between the top of the filter and the openable top.

15. The bag of claim 1 further comprising a first pull tab affixed to an exterior portion of the first side and a second pull tab affixed to an exterior portion of the second side.

16. A method of preparing a culture sample, comprising
converting a bag of claim 5 to the open configuration, thereby forming two compartments, the first compartment defined by the first side and the filter and the second compartment defined by the second side and the filter;
opening the openable top; and
placing a test sample in one of the first compartment or the second compartment such that the test sample contacts the liquid growth media.

17. The method of claim 16, further comprising removing at least some of the liquid growth medium from whichever of the first compartment or second compartment does not contain the test sample.

18. The method of claim 17, wherein the method further comprises allowing the test sample to remain in contact with the liquid growth medium for a time period sufficient for microorganisms on the test sample to form colonies in the liquid growth medium.

19. (canceled)

20. The method of claim 16, further comprising closing the openable top of the bag after placing the test sample in one of the first compartment or the second compartment.

21-24. (canceled)

25. A method of preparing a culture sample, comprising
converting a bag of claim 1 to the open configuration, thereby forming two compartments, the first compartment defined by the first side and the filter and the second compartment defined by the second side and the filter;
opening the openable top;
adding liquid growth medium to the bag; and
placing a test sample in one of the first compartment or the second compartment such that the test sample contacts the liquid growth media.
